(19) United States
(12) Patent Application Publication
(10) Pub. No.: US 2012/0203129 A1
(43) Pub. Date: Aug. 9, 2012

(54) Brain Machine Interface Device

(76) Inventor: Robert L. Rennaker, II, Norman, OK (US)

(21) Appl. No.: 13/228,256

(22) Filed: Sep. 8, 2011

Related U.S. Application Data
(63) Continuation of application No. 11/346,507, filed on Feb. 1, 2006, now abandoned.
(60) Provisional application No. 60/649,728, filed on Feb. 3, 2005.

(51) Int. Cl. A61B 5/0476

(52) U.S. Cl. 600/544

(57) ABSTRACT

A distributed real-time wireless neural interface including a reader and an array of distinct recording devices having a wireless section and a sensor section. The reader outputs and receives radio-frequency signals. The wireless section of the array includes an rf power converter, an antenna, a regulator, and a modulator. The antenna receives the radio-frequency signals output by the reader and provides the radio-frequency signals to the rf power converter which converts such radio-frequency signals to power signals. The regulator receives the power signals and regulates such power signals to provide stable power signals. The sensor section receives the stable power signals and is adapted to detect neural activity and provide output signals containing information indicative of such neural activity to the modulator. The modulator receives the power signals and the output signals from the sensor section communicates the information in the output signals to the reader via the antenna.

### CROSS-REFERENCE TO RELATED APPLICATIONS

The present patent application is a continuation of U.S. Ser. No. 11/346,507, filed on Feb. 1, 2006, which claims priority to the provisional patent application identified by U.S. Ser. No. 60/649,728, filed on Feb. 3, 2005, the entire contents of which are hereby incorporated herein by reference.

### STATEMENT REGARDING FEDERALLY SPONSORED RESEARCH AND DEVELOPMENT

Not Applicable.

### BACKGROUND OF THE INVENTION

The human nervous system encodes information using electrical signals known as action potentials (spikes) which are generated by neurons and sensory receptors. The brain is a highly organized complex structure composed of billions of neurons which integrates multimodal sensory information to control behavior. A sensor capable of providing a stable, robust connection in humans could be used to study, treat, monitor treatments or diagnose neurological conditions. A wide range of neurological conditions could benefit from such a device, including but not limited to paralysis, deafness, blindness, Parkinson's, Alzheimer's, and epilepsy.

Multi-channel neural recording interfaces have been under development for over 30 years¹. Currently the state-of-the-art in neural interfaces employs a monolithic design with 10's to 100's of recording sites on a single device. These monolithic neural interfaces are designed for recording from neocortical structures and cannot be used for structures deeper than about 5 mm. Not only does the monolithic design construct limit the depth of the interface, but there are several other structural characteristics that significantly impact their functionality and longevity.

Technologies capable of chronically interfacing with the nervous system have been under development since the 1960's. The current state-of-the-art devices have been designed to specifically interface with the neocortex for the development of neural prosthetics. A neural prosthetic is typically designed to provide a patient with sensory information or control external devices. As a result these neural interfaces are designed to interface with primary sensory or motor cortical areas. They are designed for monitoring large numbers of neural cells in a localized superficial (5 mm) cortical area. Due to the monolithic structure, these devices cannot be used to monitor deep structures or be distributed across or within the brain. Their monolithic design not only significantly limits their usefulness but have several detrimental characteristics.

Because the state-of-the-art designs incorporate 100's of recording sites on a single probe they require relatively large interconnects. The wires and connectors are a major source of failure for these devices. Techniques and methods are being developed to implement wireless devices^{2 3 4}. For these devices to be wireless the amount of power used must be minimized to avoid tissue heating and damage. Developing a neural interface with 100 s of recording sites with onboard filtering, spike detection and RF power and communication and minimizing the power usage is a daunting task. Efforts are underway to develop low-power circuitry to perform these functions.

$${f}_{c} = ( \frac{1}{2 π RC} ) \\ {f}_{c} = CutoffFrequency \\ R = Resistance \\ C = Capacitance \qquad ( 1 )$$

Several low power amplifier designs have been reported in literature^{5 6 7}. These designs incorporate band-pass filters. Depending on the particular neural interface, the design constraints significantly change. For the micro neural interface the entire circuit must be manufactured using the AMI process. The use of external capacitors is not feasible. This design uses a MOS-bipolar pseudoresistor that allows the use of small value capacitors for filter design. The filter properties of an RC circuit are a function of resistance and capacitance based on equation (1). The largest value of a resistor that can be created using standard VLSI design is 10⁶ ohms. To get a cutoff at 150 Hz the capacitance value is 1×10⁻⁹ farads. To build a capacitor of this value would require 1.25×10⁶ um². The entire chip is only 4×10⁶ um². Using the pseudoresistor design the same cutoff can be obtained with a capacitor size 125 um².

Standard neural interfaces use sampling rates of 25-30 kSamples/sec with a minimum resolution of 10 bits. A device with 100 recording channels would require a data rate of 25 to 30 Mbits/sec. The use of RF to transmit at this rate would require unsafe power levels for an implanted device. Spike detection circuits are under development to reduce the bandwidth requirements by transmitting only when a spike is detected. Transmissions of spike waveforms or spike times are possible solutions. Transmitting a unique channel ID code when a spike is detected would require the least amount of power.

Neural data is typically modeled as spiking events in a Gaussian noise background. Spikes have amplitudes up to 500 uV. Spike detection circuits are designed to detect spiking events and reject occasional spikes in the noise. For a review of spike-detection algorithms see Obeid and Wolf (2004). Several techniques have been developed including static detectors, adaptive threshold detectors, template matching, wavelets, and energy based detectors. Obeid found that maximizing the signal-to-noise ratio and taking the absolute value of the signal is the most effective means of improving spike detection not implementing complex preprocessing.

The major issue for most of the spike detections is setting the threshold correctly. The neural noise tends to be non-stationary with occasional spikes in the noise level. The threshold level should vary as a function of the noise level to optimize spike detection and noise rejection. The use of a circuit that estimates the rms level of the signal appears to be the most computationally simple and robust solution. Several designs have been reported.

Power and communication provide two of the greatest hurdles for neural interfaces. For chronic applications wires are currently being used to transmit power and communication. These wires can provide a route of infection and are a significant source of failure. Work has been performed on the design of coils for transcutaneous transmission for powering devices beneath the skin, such as cochlear implants. Very little work has focused on powering device inside the skull.

Multi-channel micro-wire designs have been used for over 50 years. These devices are typically made from 8 to 32 microwires, 50 um tungsten or stainless steel, insulated with polyimide or Teflon. The wires are typically arranged in an array pattern of 2×8 wires. The wires are implanted into the brain. The connector is then attached to the skull with acrylic. The wires can be sharpened and coated with various polymers to provide a range of impedance values.

Typically microwire arrays provide viable recordings for a month or two, however several studies have reported recordings lasting several months or more^{16 11}. These devices have provided great insight into neural processing, cortical plasticity, and neural prosthetics. Custom micro-wire designs have been designed and used since 1998. Microwires are relatively easy to manufacture, low cost and highly reliable. The drawbacks to microwire designs are that they are highly prone to motion artifact making it nearly impossible to record from behaving animals, the wires must pass through the skull and skin and they have several of the design flaws found in the monolithic structures as discussed below.

Phil Kennedy has developed a neural interface “the cone electrode”. The cone electrode is constructed from two 50 um diameter microwires inserted into a glass pipette tip. The pipette is filled with neurotrophic factors that encourage growth of the neurons into the pipette. The probes are individually inserted and positioned. Independently inserting each device allows the researcher to optimize placement, yield and signal-to-noise ratio. The size, independence and flexibility of the probes minimize tissue damage. Another benefit is that the neurotrophic factors result in neural tissue growing into the cone resulting in increases of the signal-to-noise ratio overtime and mechanically fixing the electrode in place reducing motion artifacts.

The cone electrodes have provided stable recordings in human patients for up to 16 months. The probes allow locked-in ALS patients to control a cursor on a computer screen to communicate with family and doctors. The results of these studies demonstrate that a chronic neural interface can be used to assist patients with neurological deficits.

There are major draw backs to the neurotrophic electrode. First it is hand made, and therefore cannot be manufactured in large quantities. Second, the wires must pass through the skull and skin. This design is susceptible to damaged connectors and noise from external sources.

Typical current state-of-the-art neural interfaces are displayed in FIGS. 1a and 1b. While these designs are constructed using different manufacturing techniques, they all utilize a similar monolithic design construct. As used herein, the term “monolithic” is defined as a neural interface that incorporate 10s or 100's of recording channels on a single probe. These individual channels are typically connected to a rigid platform. Shanks range from 1 to 5 mm long depending on the manufacturing process.

The Michigan probe (depicted in FIG. 1a) is constructed by assembling several planar silicon recording probes onto a single platform. Michigan uses a boron-etch-stopped silicon substrate that produces flexible thin electrodes. The probes typically have several shafts (15 um×60 um) with recording site along the lengths of the shafts. Electrode features sizes as small as 1 um can be created. The size, shape and impedance of the recording sites are optimized for specific applications ranging from 40-400 um². Efforts are currently underway to place signal conditioning, spike detection/sorting, RF power and communication and multiplexing hardware on the platform. These circuits are made using a standard p-well CMOS process. The amplifier has a gain of 40 dB with a bandwidth from 10 Hz-10 kHz and dissipates less than 100 uW. The high-pass portion of the filter removes the dc offset caused by the electrode/tissue interface. Spike amplitudes range from 50-800 uv peak-to-peak with a typical noise floor of 30 uV.

Studies on the chronic recording properties of the Michigan probe have shown that this design provided viable neural recordings for periods lasting 4 months. In another study, the mean signal-to-noise ratio was 8.55 and decreased to 6.35 over a 54 week period. Despite this success, much work remains to create devices capable of recording from humans for the remainder of their lives (decades). Efforts are underway to improve yield and longevity by incorporating drug delivery sites or coating the entire surface of the electrode with bioactive materials.

Utah uses a micromachining technique in coordination with VLSI technology to manufacture a silicon substrate multi-channel probe. A diamond dicing saw and chemical etching are used to create a monolithic structure out of a 4.2 mm×4.2 mm monocrystalline block of silicon. The diamond saw cuts 300 um deep groves on the back of the silicon block. The back, including the groves are coated with a frit sealing glass. After the glassing procedure a diamond wheel polishes the back such leaving bare silicon squares between a glass grid. The opposite side is then cut into square columns and etched forming the recording shanks. The groves between the recording shanks are cut down to the glass to ensure that the shanks are insulated from one another. The shanks then undergo an acid etch to taper and sharpen the shanks. The tip of each probe is metalized with platinum to form the recording site. Then the entire probe is coated with polyimide to insulate the shanks. Recording sites can only be placed at the tips of the shanks. Signal-to-noise ratios of 11.5 have been reported in somatosensory cortex of cats. The Utah probe has been used to record from visual cortex for 100 days.

While differing construction methods and materials are used to create the monolithic devices, they all utilize a similar monolithic design construct. Large numbers of recording shafts are incorporated onto a single platform. The monolithic structure is designed to interface with a large number of superficial neurons (<5 mm). These devices provide high density recording with 100 or more recording sites in a 2 mm×2 mm area. These devices are specifically designed to record from neocortex or peripheral nerves for control of motor prosthetics. These devices have large signal-to-noise-ratios, and provide excellent recordings for acute and short-term chronic implants. These devices perform excellent for their intended applications, but they are limited to interfacing with superficial structures.

An analysis of the monolithic designs reveals many inherent design features that limit their functionality. The micro neural interface, constructed in accordance with the present invention, is specifically designed to overcome these limitations allowing researchers greater access to neural structures. The following details those design issues that limit the functionality of current state-of-the-art neural interfaces.

Utility—The multi-channel neural interface is designed to be implanted on the surface of the brain or peripheral nerves. These devices are limited to recording from cells near the surface. These devices typically cannot record from structures deeper than 5 mm. For higher order mammals (humans, cats), this design will only allow recordings from the neocortex, peripheral nerves or other superficial structures.

Rigidity—Tens or hundreds of shafts are connected to a single platform; movement of the platform could result in motion of all of the shafts relative to the neural tissue. This motion could result in injury to the surrounding tissue including neurons and blood vessels. Overtime, it would be expected that considerable damage would be induced around the implant site resulting in a loss of neural information.

Surface Implant—The monolithic designs use a platform with shafts extending off of the bottom. This design requires that the interface be implanted at the surface of the structure. Violent head movements could result in the probe striking the inside of the skull, damaging cortical tissue and the device.

Robustness—Because the devices physically cannot overlap, failure of the probe would result in loss of all data from that neural population.

Yield—The rigid structure does not allow the recording sites to be independently positioned. All of the recording sites are inserted simultaneously. After insertion, the individual channels cannot be repositioned to optimize recording properties. It is likely that some portion of the recording sites will not provide viable neural recordings.

Complexity—These designs are highly complex and require a large number of interconnects. The large number of interconnects and complexity provide a large number of potential failure points. Failure at critical points could result in a loss of all data from that neural interface.

Thus, there is a need for an impoved neural interface. It is to such an improved neural interface that the present invention is directed.

### BRIEF DESCRIPTION OF THE SEVERAL VIEWS OF THE DRAWINGS

So that the above recited features and advantages of the present invention can be understood in detail, a more particular description of the invention, briefly summarized above, may be had by reference to the embodiments thereof that are illustrated in the appended drawings. It is to be noted, however, that the appended drawings illustrate only typical embodiments of this invention and are therefore not to be considered limiting of its scope, for the invention may admit to other equally effective embodiments.

FIG. 1a is a perspective view of a prior art monolithic design for a neural interface.

FIG. 1b is a perspective view of another prior art monolithic design for a neural interface.

FIG. 2 is a diagrammatic, schematic view of a distributed real-time wireless neural interface, constructed in accordance with the present invention.

FIG. 3 is a block diagram of an exemplary recording probe constructed in accordance with the present invention.

FIG. 4 is a schematic diagram of an exemplary interface of a sensor section of the recording probe.

FIG. 5 includes three charts showing the operation of the interface of the sensor section of the recording probe.

FIG. 6 is a schematic diagram of an exemplary RF-DC converter circuit.

FIG. 7 are graphs showing power consumption as a function of the number of stages used in the RF-DC converter circuit.

FIG. 8a is a schematic view of an exemplary rf power converter.

FIG. 8b is a graph illustrating the power input to the RF circuits.

FIG. 9 is a schematic diagram of an exemplary regulator circuit.

FIG. 10 is a graph illustrating the encoding of a unique identification code into the rf signal, and the decoding of the rf signal to obtain the unique identification code.

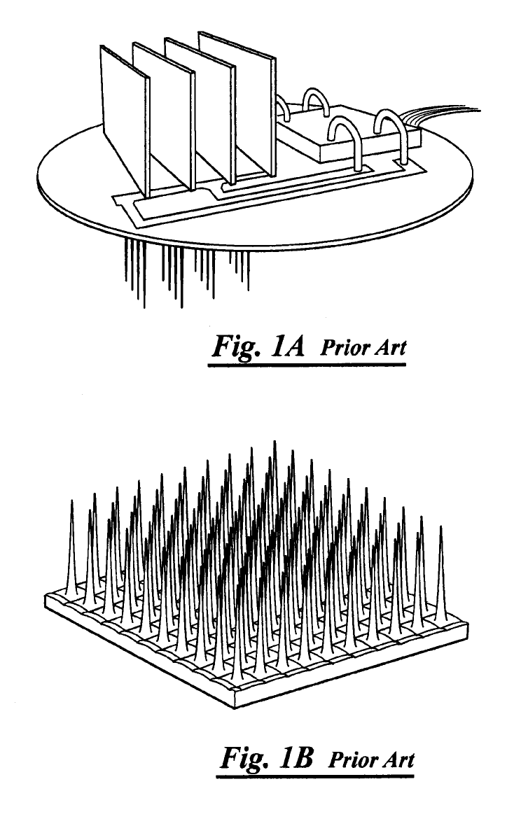

### DETAILED DESCRIPTION OF THE INVENTION

Presently preferred embodiments of the invention are shown in the above-identified figures and described in detail below. In describing the preferred embodiments, like or identical reference numerals are used to identify common or similar elements. The figures are not necessarily to scale and certain features and certain views of the figures may be shown exaggerated in scale or in schematic in the interest of clarity and conciseness.

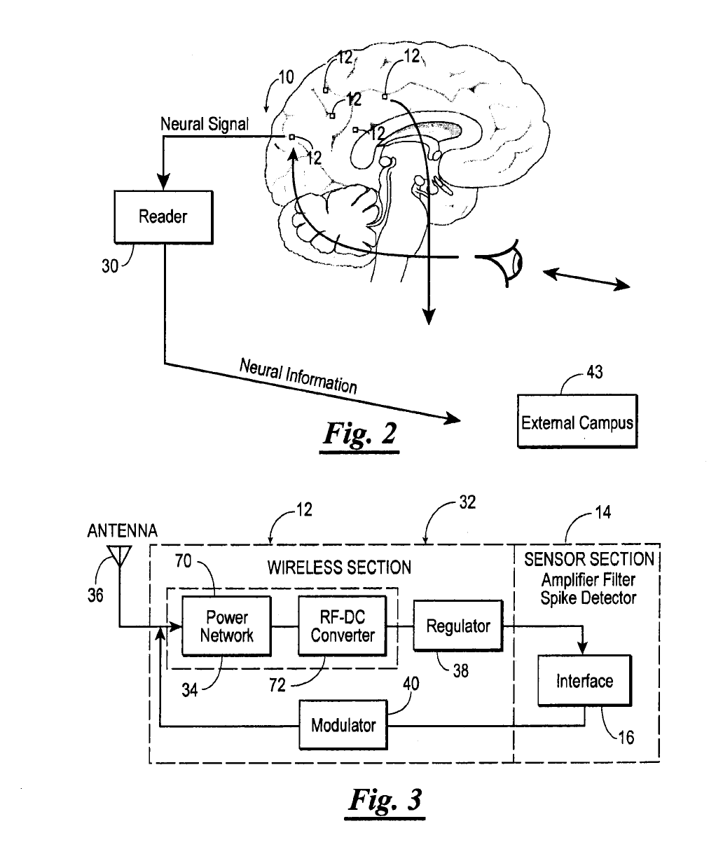

Referring now to the drawings and in particular to FIG. 2, shown therein and designated by a reference numeral 10 is a distributed real-time wireless neural interface constructed in accordance with the present invention. The neural interface 10 is capable of overcoming the limitations inherent in current state-of-the-art monolithic neural interfaces. The neural interface 10 includes a distributed array of independent wireless single-channel extracellular recording probes 12. Only five of the probes 12 are shown for purposes of brevity. Each individual probe 12 is equipped with an onboard sensor section 14 (FIG. 3) preferably including an interface 16 with signal conditioning and spike detection circuitry 18 (FIG. 4) to facilitate low-power, high-throughput monitoring of neural networks, such as cortical networks.

The neural interface 10 will be described in conjunction with a particular preferred application, i.e., the use of the neural interface 10 for reading neural signals in a brain 20 (FIG. 2). However, it should be understood that the neural interface 10 can be used for reading neural signals in other parts of a body.

The size and design of the recording probes 12 allows the recording probes 12 to be independently implanted in deep structures throughout the brain 20 and to be powered and communicate via an RF link. The probes 12 will preferably be designed and manufactured using standard photolithography techniques allowing them to be mass produced with high quality and precision.

In general, the neural interface 10 is provided with a reader 30, as well as the array of distinct neural recording probes 12. The reader 30 outputs and receives radio-frequency signals to power the recording probes 12 as well as to communicate with the recording probes 12. At least two of the recording probes 12 (and preferably all of the recording probes 12) are provided with a wireless section 32, as well as the sensor section 14 discussed above. The wireless section 32 is provided with an rf power converter 34, an antenna 36, a regulator 38, and a modulator 40.

The rf power converter 34 converts the radio frequency signals into power signals for powering the remaining circuitry in the wireless section 32, as well as the interface 16 of the sensor section 14. Typically, the rf power converter 34 converts the radio frequency signals into DC signals, however, it should be understood that the rf power converter 34 can convert the radio frequency signals into any power signal capable of powering the powering the remaining circuitry in the wireless section 32, as well as the interface 16 of the sensor section 14, such as AC signals.

The antenna 36 receives the radio-frequency signals output by the reader 30 and provides the radio-frequency signals to the rf power converter 34 wherein the rf power converter 34 converts such radio-frequency signals to power signals as discussed above. The antenna 36 is preferably a far field antenna, however, it should be understood that the antenna 36 could be constructed in other manners, and several different geometries will be tested to minimize SAR and maximize efficiency. Furthermore, the antenna 36 could be formed of a single antenna or multiple antennas.

The regulator 38 receives the power signals and regulating such power signals to provide stable power signals; The regulator 38 can be any type of circuitry which regulates the power signals to provide stable power signals typically above the threshold voltage of the signal conditioning and spike detection circuitry 18, and also operates within the power constraints of the recording probes 12.

The modulator 40 receives the power signals and is in communication with the antenna 36 for utilizing the antenna 36 to communicate with the reader 30. The modulator 40 is preferably a communication device designed for wireless communications, and that utilizes a small amount of power (e.g., <200 uW). The modulator 40 can utilize any suitable modulation technique, such as PSK, spread spectrum, PWM, ASK or the like.

The sensor section 14 receives the stable power signals. The sensor section 14 is adapted to detect neural activity, such as by having a copper plate to detect differences in voltage levels, and is also adapted to provide output signals containing information indicative of such neural activity to the modulator 40 of the wireless section 32 whereby the modulator 40 communicates the information in the output signals to the reader 30.

The reader 30 external device sends out the rf signal and polls each recording probe 12 to determine if a spike was detected. The reader 30 reads the neural information transmitted by the recording probe 12 and passes such neural information to an external computer 43 for further processing.

The neural interface 10 is designed to allow real-time monitoring of a wide range of locations and structures in the nervous system in behaving animals that cannot be obtained from standard micro-wire or state-of-the-art neural interfaces. The neural interface 10 will allow researchers to study complex processing of information and how those processes are altered by learning, disease, or medical treatments. At the same time this technology could eventually be used in neural prosthetic systems to assist paralyzed patients. The neural interface 10 has the potential to significantly improve the neurological healthcare of our state and nation.

Micro Neural Interface Design Concept:

The neural interface 10 is designed to avoid problems inherent with monolithic neural interface designs. The design of the neural interface 10 employs lessons learned from the neurotrophic electrode and current state-of-the-art neural interfaces. The neural interface 10 is designed with the distributed array of independent recording probe 12 capable of recording from multiple structures and in multiple locations in a host of animal models including rats and mice. The following is a list of features for the neural interface 10 to meet this goal.

The Neural Interface 10 has the following features:
Non-rigid platform—the recording probes 12 form an array of independent channels;
The chip design for the recording probes 12 must remain as small as possible (i.e., sub-millimeter scale);
The entire chip for each recording probe 12 must use minimal power (<200 uW/probe);
Onboard low-power spike detection circuitry;
Dynamic threshold detector for spike detection circuitry;
The recording probes 12 form independently positionable channels;
The recording probes 12 are capable of recording from deep biological structures; and
The recording probes 12 are typically constructed as integrated circuits which can be mass produced.

Wireless Power and Communication System

A far field (antenna) topology for power and data transfer is most appropriate for this application. As overall size of the recording probe 12 is critical, using a high frequency link allows for smaller and hence, more efficient coupling structures. In fact, simple ½ and ¼ wave dipole and monopole antennas fall well within the sizing requirements. Furthermore, the use of high frequency allows for high data rates, which will be required if multiple recording probes 12 are implanted. Finally, the infrastructure required for high frequency power and communication scales rather nicely given the sizing requirements. For example, filtering of the ripple associated with RF to DC power conversion at ˜100 KHz would require a capacitor on the order of 10 s of mF, hardly realizable as a monolithic structure on silicon for the sizing contemplated here, while at a frequency of 2.45 GHz, the filtering capacitor will reduce to 10 s of pF. To be sure, high frequency topology brings with it its own unique technological issues such as the effects of parasitics, environment, layout, and generally low RF to DC conversion efficiency of active components, but high frequency far field topology provides a more robust design space in which to contemplate possible solutions, than the mutual inductance approach.

Approach

A block diagram of the recording probe 12 is shown in FIG. 3. The micro neural interface design concept is the direct result of attempting to fulfill these requirements. Specifically, the neural interface 10 includes the recording probes 12 which are batch fabricated providing high volume and high quality. Each recording probe 12 can be independently positioned to optimize recording quality and yield. The recording probes 12 can be implanted in cortical and sub-cortical structures. Failure of any single probe 12 should not affect the operation of the remaining probes 12. The recording probe 12 can have a housing coated in various biomaterials or the recording probe 12 can have a housing constructed of biomaterial to optimize the neural interface. Finally, the onboard signal conditioning and spike detection circuitry 18 will allow researchers to perform high-throughput analysis in behaving animals including rats and mice.

Amplifier Development

Minimize Power

The long-term objective is to wirelessly power the probes 12 via the reader 30 using an external RF source. To realize this goal, the power used by each of the probes 12 must be minimized. For VLSI designs power consumption is a function of several factors including field effect transistor (FET) sizes, process size, number of structures. For each of the design iterations a major focus will be on minimizing the amount of power used by optimizing the transistor design, and minimizing circuit complexity.

Minimize Noise

For the recording probe 12 to detect neural activity it must have a large signal-to-noise ratio (SNR). Most commercially available neural interface systems incorporate a band-pass filter from ˜300 Hz to ˜5 kHz to reduce noise. However, implementing a band-pass filter in standard VLSI technology requires large foot print capacitors and cannot be implement in AMI 0.6 um technology and still meet the sub-millimeter size requirements for this design. However, non-standard designs have been reported that would allow filtering, and these options will be explored.

Test Spike Detection Circuit

The purpose of the recording probe 12 is to record when neural events (spikes) occur. A single probe 12 can record from multiple cells. Spike sorting allows the information from the individual cells to be analyzed separately, typically providing more information about neural coding than single units or unsorted multi-unit data. However, spike sorting typically requires significant computational power and implementing an automated spike sorter in not feasible given the power and size constraints for the micro neural interface. As a result, this specific aim will develop a circuit that detects spiking events.

Spike Detection Circuit

The spike detection circuit 18 is low power, with a small foot-print, and highly accurate in identifying neural spikes.

Dynamic Threshold Circuit

The spike detection circuit 18 requires that a threshold value be set. Because the threshold will depend upon the noise level, and the noise is partially a function of recording site impedance, location and implantation duration, developing a dynamic threshold that is a function of the background activity of the signal is the most attractive solution.

Each probe 12 is designed to transmit a unique address each time a spike is detected. Testing is accomplished by including two outputs on the chip forming the recording probe 12: one for raw data, and one for the spike detector 18 to allow a comparison of the spike detector output and raw waveform. The number of spikes detected can be compared to the number of spikes present.

The spike detection circuit 18 includes a window comparator 50, a filter 52, and a switch 54 (FIG. 4). The window comparator 50 sets the switch 54 to a predetermined state, e.g., “high” when the voltage crosses the positive. The critical feature for the spike detector 18 is the selection of the threshold value. Spikes are typically on the order of 70 my to 500 my depending on distance from the process and impedance of the electrode. Setting the threshold too high or too low can be problematic. If the threshold is too low, the probe 12 will continuously be transmitting noise. If the threshold is set too high, neural activity will not be detected and transmitted.

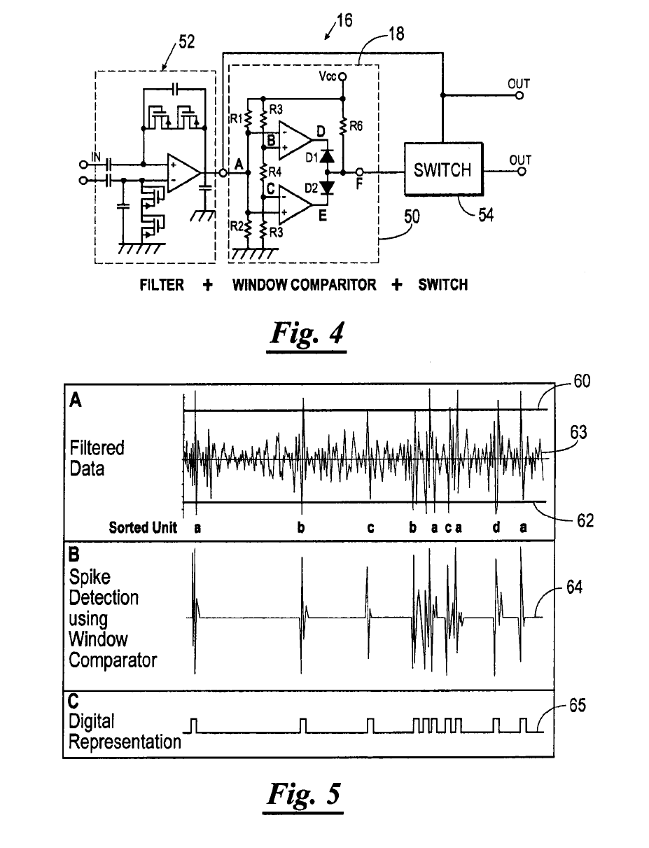

The current recording probe 12 (constructed using VLSI technology) optionally includes an input (not shown) for manually setting the threshold via an external source. This design provides an opportunity to evaluate different threshold settings as well as an external dynamic threshold circuit. FIG. 5 (including panels a, b and c) illustrate how the spike detection circuitry 18 is designed. FIG. 5, panel a shows a raw recording 63 from a Piriform cortex in a rat. The two black solid lines 60 and 62 represent the threshold settings. The letters a, b, c, b, a, c, a, d and a label the individual spikes. FIG. 5, panel b shows a waveform 64 illustrating the effect of the window comparator 50 on the raw data. Only supra-threshold values trigger a 1 ms transmission of data. FIG. 5, panel c, is a digital representation 65 of the output of the window comparator 50. The design transmits a bit high when a spike is detected, but does not sort the different waveform shapes.

Spike Detection Circuits

Determine an Optimum Threshold Value for a Fixed Threshold Detector

The spike detection circuits 18 will be designed to provide the raw output as well as the spike detector output. The performance of the fixed threshold will be evaluated using an artificial spike generator (TDT). Both outputs of the circuit will be connected to a neural recording system. The spike rate, noise floor, signal-to-noise ratio, and statistical distribution of the noise will be systematically altered with the spike generator. The number of spiking events will be compared between the raw data recording and the spike detector output channels. A 95% correct detection and less than 5% false alarm rates will be considered a successful test.

Develop and Evaluate Several Circuits to Measure the Noise Level

The dynamic threshold can be tested using waveforms from an artificial spike generator and raw data obtained from chronically implanted rats will be used. The spike rate, noise floor, signal-to-noise ratio, and statistical distribution of the noise will be systematically altered with the spike generator to evaluate the different circuit designs.

The filter 52 is preferably a bandpass filter passing signals having a frequency between about 500 Hz to about 4000 Hz. The filter 52 is used to reduce noise and amplify the signal for the spike detector circuitry 18. The switch 54 is set to the predetermined level when a spike is detected.

RF Power Converter

The RF power converter 34 takes the applied power as received by the antenna 36, and converts the non-modulated RF signal into a DC voltage. This DC voltage is output to the regulator 38 so that a constant DC voltage is applied to the interface 16 of the sensor section 14 (e.g., the signal conditioning circuitry (amplifier, filter, and spike detector)) regardless of the power fluctuations as received by the antenna 36. Once a spike has been detected the data is sent to the modulator 40 where it is appropriately converted to a modulated RF signal which goes back out the antenna 36 to the reader 30 for data capture, analysis, and end usage.

Antenna

The antenna 36 serves two functions: (1) efficiently capturing the applied RF signal for conversion to DC as well as (2) having enough bandwidth for high data rate and modulated signals to pass between the recording probe 12 and the reader 30. In order to estimate sizing of the antenna 36 it is necessary to know the dielectric properties of the brain material in which the antenna 36 will be implanted. Typical measured results indicate that the dielectric permittivity of grey matter is between 40 and 50 at 2450 MHZ. Using an average er of 45, the wavelength at 2450 MHz is which is more than reasonable for an implanted antenna length. Of equal importance is the attenuation of the body at the frequency of choice. Recently, a measured attenuation of −30 dB was achieved for a distance of 25 mm in an intraocular implanted device operating at 2450 MHz using a slot/patch antenna arrangement. It is therefore certain that both simulation and experimentation will be required in order to balance and tradeoff between the length and topology of the antenna 36 and the body (skull/brain) attenuation that the signal will experience. Additionally, it seems appropriate to place the antennas 36 as close to the skull as possible in order to minimize the RF link distance, as well.

Power Match

The rf power converter 34 includes a power match network 70 and a RF-DC converter 72. The power match network 70 matches the power between the antenna 36 and the RF-DC converter 72 to maximize the power transfer between these components. The power match network 70 is typically an LC network of relatively high Q so that only reactive power is dissipated in the network. Additionally, the high Q of the match typically has a natural bandpass characteristic so that the network doubles as a filter, which reduces the overall input noise to the RF power converter 34.

RF Power Converter

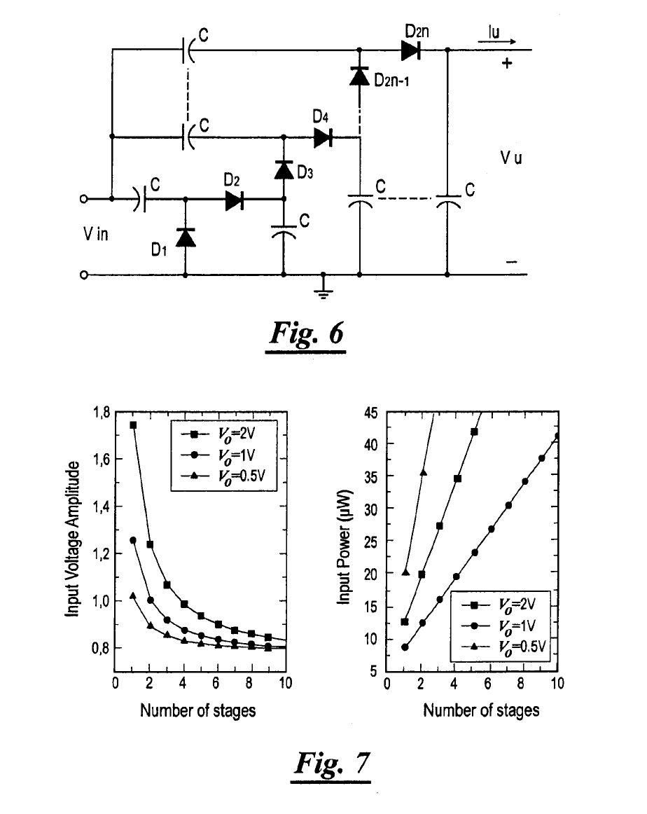

In one preferred embodiment, the rf power converter 34 is arranged into a multiplier rectifier arrangement as shown in FIG. 6. The multiplier converter both multiplies Vin to a final value of Vu as well as converts Vin (RF) to Vu (DC). The canonical converter is the circuit represented by C, D1 and D2. The diodes, by their very nature, perform RF to DC conversion as they serve as peak power detectors. By having D1 and D2 arranged as shown the output voltage as seen across C between D2 and D3 is double what would be achieved using only a single voltage. Choosing C large enough to act as a short for the RF signal, only the DC component of the voltage remains on C. D3 and D4 also perform the same RF to DC conversion, however, since C already has a DC voltage on it from the first stage, any newly converted RF to DC voltage will simply add to it. The same reasoning can be used for each subsequent stage in order to get the desired Vu. However, one can easily see that the parasitics of the diodes such as stray capacitance, reverse leakage currents, etc will be a limiting factor so that the multiplier approaches an asymptotic value rather than an ever increasing output voltage. The transcendental equation describing this behavior can be derived from first principles and is given by Equation 2:
where N is the number of diode multiplier stages, Iu is the load current, Vu is the output voltage of the multiplier, BO is the Bessel function of the argument (Vo/Vt), and Vo is the output voltage of each diode. Clearly as N increases an asymptotic function is described. This is seen in FIG. 7, which is a plot of this equation for both input voltage and input power.

$$( 1 + \frac{{I}_{U}}{{I}_{S}} ) exp ( \frac{{V}_{U}}{2 N {V}_{t}} ) = {B}_{0} ( \frac{{V}_{0}}{{V}_{T}} ) \qquad ( 2 )$$

Notice from FIG. 7 that although a smaller input voltage can be used in combination with more stages to generate a given Vu, the power required to be supplied to the multiplier must increase to compensate for diode losses and parasitics. Hence, a judicious balancing of input voltage, power, and stages must be undertaken to maximize the RF-DC conversion.

The recording probe 12 will operate at approximately 1.2V and consumes approximately 0.2 mW of power. Let us assume that all the additional circuitry, mismatches, and parasitics increase the power dissipation by 50%. Therefore, we need to supply at the input of the rf power converter 34 0.3 mW. In RF terms this is −5 dBm of power, with an input voltage of 0.122 V for a 50 W system impedance. We have simulated such a multiplier/converter with up to 3 stages (N=3), with the results shown in FIG. 7.

Using Schottky diodes we see that the N=2,3 topology will supply a voltage that is greater than 1.2V. Also notice that the capacitors used are 10 pF. Given the AMI process, a 10 pF capacitor will only occupy ˜100 mm on a side. Hence going to a high frequency rather than a coupled coil approach provides the benefit of size reduction for the active circuitry.

Regulator

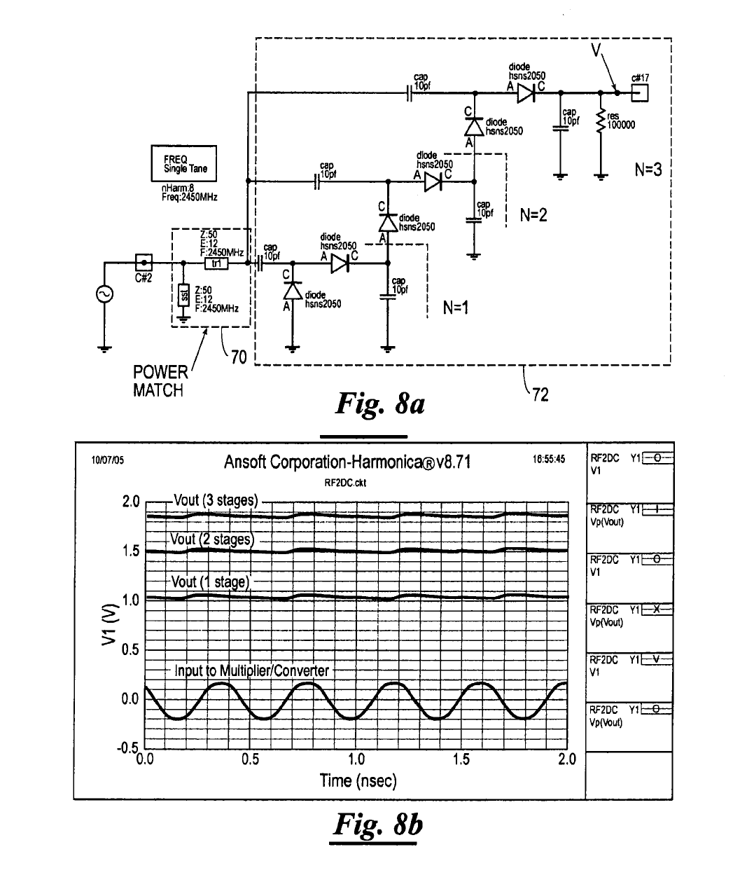

From FIG. 8a it can be seen that the rectified voltage has some minor ripple. We can always trade ripple for output capacitance. By increasing the on chip capacitance, the ripple is reduced however the capacitance area is increased thereby decreasing chip real estate for other functionality. By decreasing the capacitance, the ripple is increased however less on chip real estate is required. FIG. 8b shows the waveform staying above the operational value. The number of stages can be traded off to minimize the oscillation for increased voltage with fewer stages.

However it is important to realize that as long as the ripple is such that it is above the dropout limit of the regulator 38, the regulator 38 will still maintain the output voltage of 1.2V. Hence the regulator 38 is designed with minimum overhead so as to allow the ripple to be as large as necessary. As an example, if the recording probe 12 requires 1.2V and the dropout voltage of the regulator is 0.1V then the peak ripple+rectified DC voltage cannot drop below 1.3V. Another key aspect of the regulator 38 is that it's own quiescent current must be very low so as not to tap the power we want for the recording probe 12. Numerous regulator designs exist for the BiCMOS process.

Modulator

There are numerous ways of wirelessly communicating the data from the recording probe 12 to the reader 30. However, they typically fall into two categories (1) active and (2) passive. Active techniques require an on-board oscillator that actively sends out the signal that has the desired data embedded in the carrier waveform. Of the passive techniques, backscattering of the incident RF used to generate power is the preferred method. There are two methods of backscatter that are preferred, ASK and PSK. Amplitude Shift Keying (ASK) allows the real part of the impedance of the antenna 36 to be modulated, while PSK allows the reactive part of the impedance of the antenna 36 to be modulated. Both methods will be evident at the reader 30 as either a change in amplitude of the received signal (ASK) or a change in phase of the received signal (PSK). However, ASK necessarily requires that the power to the RF power converter 34 is not constant since changing the real part of the antenna's impedance in essence makes the antenna 36 selectively either a good acceptor/transmitter of RF or not. Hence, not only does the ripple effect performance, we also must insure that the modulation depth of the ASK signal does not force the rectified voltage to drop below the level of the dropout of the regulator 38. Additionally, changing of amplitude on the power input side runs the risk of producing glitches in the recording probe 12 that could corrupt the data from the neurons—this remains to be determined. A better approach would be to allow for constant power, i.e. constant input amplitude of the RF signal into the converter and transmission of the RF to the receiver. PSK allows for this as only the phase of the received signal is being modulated and not the amplitude. Hence, glitching, regulator dropout, etc. effects become drastically reduced.

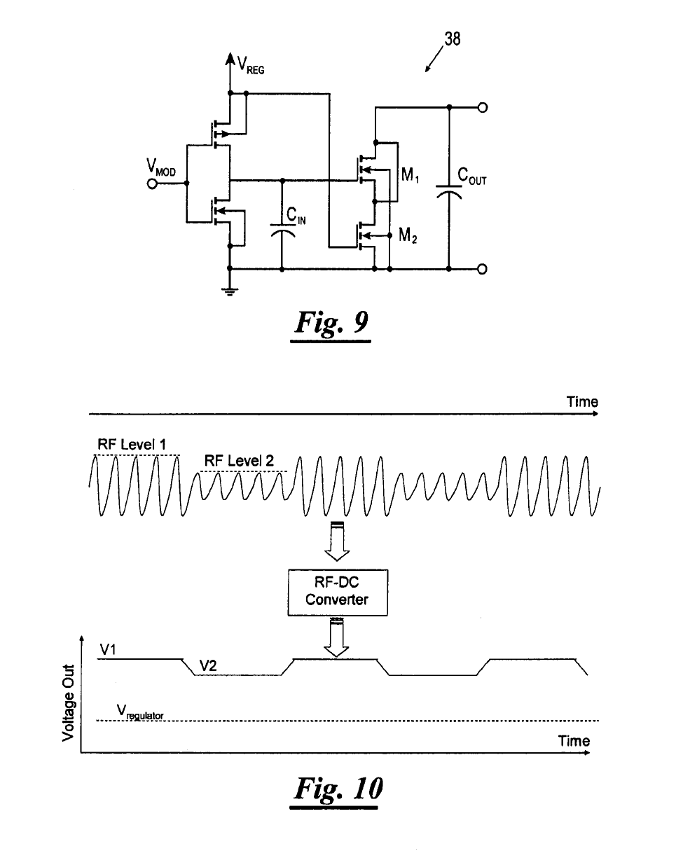

For illustrative purposes, a typical PSK modulator is shown in FIG. 9. Vmod is the signal from the interface 16 of the sensor section 14, Vreg is the regulated DC voltage powering the modulator 40, and Cout in parallel with M1 and M2 provide for the variable capacitance that modulates the reactive part of the antenna 36. M1 is used simply as a variable capacitor. The overall modulator consumes 15 nW of power. Detailed operational explanation of this representative circuit can be found in [8].

System Architecture

Embedding ID Information in RF Power Signal

The operation of the neural interface will now be described. Many of the recording probes 12 can be interrogated and the combined output can be read and monitored by the reader 30. The key, of course, is accessing each individual recording probe 12 when up to 100 or more other probes are implanted and monitored simultaneously.

Recall that the regulator 38 sets the DC level at which the recording probe 12 will operate. Hence as long as a DC voltage is provided that is above the regulators threshold level, the recording probe 12 will be powered at a constant voltage level. By having the DC level somewhat higher than the required threshold level, the incoming RF signal is modulated to embed identification information into the power signal produced by the rf DC converter 72 of the rf power converter 34. This is shown conceptually in FIG. 10. If a bi-level RF signal is purposely transmitted to the recording probe 12, the RF-DC converter will produce a DC output waveform that reflects this bi-level RF signal. By letting V1 be considered a “1” and V2 a “0” binary information is provided on the output of the RF-DC converter. Provided both V1 and V2 are above the regulator's V threshold, the regulator 38 powers the recording probe 12 uninterrupted while the binary signal will contain the ID of the recording probe 12. This ID voltage signal is then appropriately scaled via resistor divider and directly passed into the spike detection circuitry of the interface 16 of the recording probe 12. If the state engine on the recording probe 12 determines that the ID voltage matches the recording probe 12's internal ID, the recording probe switches on and transmits its data via the aforementioned PSK backscatter methodology for a TBD time of transmission. Should the ID not match, i.e. another recording probe 12 is being queried, the recording probe 12 remains off and no data is transmitted. In this way, the entire ensemble of recording probe 12 probes can be polled to acquire their data so that no individual recording probe 12 conflicts with any other. Additionally, the probability of external RF interfering with the recording probe 12 operation is reduced drastically. This is seen because (1) only external RF of the frequency band chosen will have an effect due to the bandpass filtering of both the antenna 36 and power match 70 circuitry, and (2) will only effect the recording probe 12 when it is trying to determine if the ID it is receiving is correct. Provided the recording probe 12 state engine is fast and the ID signal is short in duration, only a very small time probability of interference is possible. Hence, the RF signal powers the recording probe 12 as well as interrogates a particular recording probe 12 based upon its internal ID.

This description is intended for purposes of illustration only and should not be construed in a limiting sense. The scope of this invention should be determined only by the language of the claims that follow. The term “comprising” within the claims is intended to mean “including at least” such that the recited listing of elements in a claim are an open group. “A,” “an” and other singular terms are intended to include the plural forms thereof unless specifically excluded.

### REFERENCES

1. Wise, K. D., Angell, J. B., and Starr, A. An integrated-circuit approach to extracellular microelectrodes. IEEE Trans Biomed Eng 17, 238-47 (1970).
2. Obeid, I., Nicolelis, M. A., and Wolf, P. D. A multichannel telemetry system for single unit neural recordings. J Neurosci Methods 133, 33-8 (2004).
3. Hijazi, N., Krisch, I., and Hosticka, B. J. Wireless power and data transmission system for a micro implantable intraocular vision aid. Biomed Tech (Berl) 47 Suppl 1 Pt1, 174-5 (2002).
4. Wise, K. D., Anderson, D. J., Hetke, J. F., Kipke, D. R., and Najafi, K. Wireless implantable microsystems: High-density electronic interfaces to the nervous system. Proceedings of the Ieee 92, 76-97 (2004).

### Notes: Review.

5. Obeid, I., Nicolelis, M. A., and Wolf, P. D. A low power multichannel analog front end for portable neural signal recordings. J Neurosci Methods 133, 27-32 (2004).
6. Harrison, R. R. and Caameron, C. A Low-Power Low-Noise CMOS Amplifier for Neural Recording Applications. IEEE Journal of Solid-State Circuits 38, 958-965 (2003).
7. Obeid, I., Morizio, J. C., Moxon, K. A., Nicolelis, M. A., and Wolf, P. D. Two multichannel integrated circuits for neural recording and signal processing. IEEE Trans Biomed Eng 50, 255-8 (2003).
8. Obeid, I. and Wolf, P. D. Evaluation of spike-detection algorithms for a brain-machine interface application. IEEE Trans Biomed Eng 51, 905-11 (2004).
9. Harrison, Reid R. A Low-Power Integrated Circuit for Adaptive Detection of Action Potentials in Noisy Signals. In Proc. 2003 Intl. Conference of the IEEE Engineering in Medicine and Biology Society. 2003.
10. Watkins, Paul T., Santhanam, Gopal, Shenoy, Krishna V., and Harrison, Reid R. Validation of Adaptive Threshold Spike Detector for Neural Recording. Proceedings of to 26th Annual International conference of the IEEE EMBS.
11. Porada, I., Bondar, I., Spatz, W. B., and Kruger, J. Rabbit and monkey visual cortex: more than a year of recording with up to 64 microelectrodes. Journal of Neuroscience Methods 95, 13-28 (1931).

### Notes: Article.

12. Williams, IC., Rennaker, R. L., and Kipke, D. R. Long-term neural recording characteristics of wire microelectrode arrays implanted in cerebral cortex. Brain Res Brain Res Protoc 4, 303-13 (1999).
13. Rennaker, R. L., Ruyle, A. M., Street, S. E., and Sloan, A. M. An economical multi-channel cortical electrode array for extended periods of recording during behavior. J Neurosci Methods 142, 97-105 (2005).
14. Loeb, G. E., Peck, R. A., and Martyniuk, J. Toward the ultimate metal microelectrode. J Neurosci Methods 63, 175-83 (1995).
15. deCharms, R. C., Blake, D. T., and Merzenich, M. M. A multielectrode implant device for the cerebral cortex. J Neurosci Methods 93, 27-35 (1999).
16. Schmidt, E. M., Bak, M. J., and McIntosh, J. S. Long-term chronic recording from cortical neurons. Exp Neurol 52, 496-506 (1976).
17. Taylor, D. M., Tillery, S. I., and Schwartz, A. B. Information conveyed through brain-control: cursor versus robot. IEEE Trans Neural Syst Rehabil Eng 11, 195-9 (2003).
18. Taylor, D. M., Tillery, S. I., and Schwartz, A. B. Direct cortical control of 3D neuroprosthetic devices. Science 296, 1829-32 (2002).
19. Georgopoulos, A. P., Schwartz, A. B., and Kettner, R. E. Neuronal population coding of movement direction. Science 233, 1416-9 (1986).
20. Schwartz, A. B., Moran, D. W., and Reina, G. A. Differential representation of perception and action in the frontal cortex. Science 303, 380-3 (2004).
21. Kennedy, P. R., Kirby, M. T., Moore, M. M., King, B., and Mallory, A. Computer control using human intracortical local field potentials. IEEE Trans Neural Syst Rehabil Eng 12, 339-44 (2004).
22. Kennedy, P. R., Bakay, R. A., Moore, M. M., Adams, K., and Goldwaithe, J. Direct control of a computer from the human central nervous system. IEEE Trans Rehabil Eng 8, 198-202 (2000).
23. Kennedy, P. R., Mirra, S. S., and Bakay, R. A. The cone electrode: ultrastructural studies following long-term recording in rat and monkey cortex. Neurosci Lett 142, 89-94 (1992).
24. Kennedy, P. R. and Bakay, R. A. Restoration of neural output from a paralyzed patient by a direct brain connection. Neuroreport 9, 1707-11 (1998).
25. Hoogerwerf, A. C. and Wise, K. D. A three-dimensional microelectrode array for chronic neural recording. IEEE Trans Biomed Eng 41, 1136-46 (1994).
26. Bai, Q., Wise, K. D., and Anderson, D. J. A high-yield microassembly structure for three-dimensional microelectrode arrays. IEEE Trans Biomed Eng 47, 281-9 (2000).
27. Wise, K. D. and Najafi, K. Microfabrication techniques for integrated sensors and microsystems. Science 254, 1335-42 (1991).
28. Hetke, J. F., Lund, J. L., Najafi, K., Wise, K. D., and Anderson, D. J. Silicon ribbon cables for chronically implantable microelectrode arrays. IEEE Trans Biomed Eng 41, 314-21 (1994).
29. BeMent, S. L., Wise, K. D., Anderson, D. J., Najafi, K., and Drake, K. L. Solid-state electrodes for multichannel multiplexed intracortical neuronal recording. IEEE Trans Biomed Eng 33, 230-41 (1986).
30. Vetter, R. J., Williams, J. C., Hetke, J. F., Nunamaker, E. A., and Kipke, D. R. Chronic neural recording using silicon-substrate microelectrode arrays implanted in cerebral cortex. IEEE Trans Biomed Eng 51, 896-904 (2004).
31. Kipke, D. R., Vetter, R. J., Williams, J. C., and Hetke, J. F. Silicon-substrate intracortical microelectrode arrays for long-term recording of neuronal spike activity in cerebral cortex. IEEE Trans Neural Syst Rehabil Eng 11, 151-5 (2003).
32. Campbell, P. K., Jones, K. E., and Normann, R. A. A 100 electrode intracortical array: structural variability. Biomed Sci Instrum 26, 161-5 (1990).
33. Jones, K. E., Campbell, P. K., and Normann, R. A. A glass/silicon composite intracortical electrode array. Ann Biomed Eng 20, 423-37 (1992).
34. Campbell, P. K., Jones, K. E., Huber, R. J., Horch, K. W., and Normann, R. A. A Silicon-Based, 3-Dimensional Neural Interface—Manufacturing Processes for an Intracortical Electrode Array. Ieee Transactions on Biomedical Engineering 38, 758-768 (1991).

### Notes: Article.

35. Normann, R. A., Maynard, E. M., Rousche, P. J., and Warren, D. J. A neural interface for a cortical vision prosthesis. Vision Res 39, 2577-87 (1999).
36. Rousche, P. J. and Normann, R. A. Chronic recording capability of the Utah Intracortical Electrode Array in cat sensory cortex. J Neurosci Methods 82, 1-15 (1998).
37. Rousche, P. J. and Normann, R. A. Chronic intracortical microstimulation (ICMS) of cat sensory cortex using the Utah Intracortical Electrode Array. IEEE Trans Rehabil Eng 7, 56-68 (1999).
38. Donoghue, J. P. Connecting cortex to machines: recent advances in brain interfaces. Nature Neuroscience 5, 1085-1088 (2002).

### Notes: Review

Suppl. S.

What is claimed is:

1. A distributed real-time wireless neural interface, comprising:
a reader outputting and receiving radio-frequency (rf) signals; and
an array of distinct recording devices, at least two of the recording devices comprising:
a wireless section comprising:
an rf power converter for converting radio-frequency signals into power signals;
an antenna receiving the radio-frequency signals output by the reader and providing the radio-frequency signals to the rf power converter wherein the rf power converter converts such radio-frequency signals to power signals;
a regulator receiving the power signals and regulating such power signals to provide stable power signals; and
a modulator receiving the power signals and in communication with the antenna for utilizing the antenna to communicate with the reader;
a sensor section receiving the stable power signals and adapted to detect neural activity and provide output signals containing information indicative of such neural activity to the modulator of the wireless section whereby the modulator communicates the information in the output signals to the reader.

2. The distributed real-time wireless neural interface of claim 1, wherein the antenna is characterized as a far field antenna.

3. The distributed real-time wireless neural interface of claim 1, wherein the sensor section compares the detected neural activity with a threshold value to detect spikes occurring in the neural activity.

4. The distributed real-time wireless neural interface of claim 3, wherein the sensor section includes circuitry for dynamically changing the threshold value.
